(19) United States
(12) Patent Application Publication
(10) Pub. No.: US 2025/0146931 A1
(43) Pub. Date: May 8, 2025

(54) SYSTEM AND METHOD FOR AUTHENTICATING AND CLASSIFYING PRODUCTS USING HYPER-SPECTRAL IMAGING

(71) Applicant: BottleVin, Inc., Santa Clara, CA (US)

(72) Inventor: Leslie Young HARVILL, Olympia, WA (US)

(21) Appl. No.: 18/922,979

(22) Filed: Oct. 22, 2024

Related U.S. Application Data
(60) Provisional application No. 63/547,070, filed on Nov. 2, 2023.

(51) Int. Cl. G01N 21/31; G01N 33/14; G01N 33/18

(52) U.S. Cl. CPC G01N 21/31; G01N 33/146; G01N 33/18; G01N 2201/062

(57) ABSTRACT

A system and method for authenticating and classifying products using hyper-spectral imaging is disclosed. In some implementation, the method comprises: illuminating a sample material with light emitted from a plurality of light-emitting diodes (LEDs); collecting spectra of light reflected, transmitted, or emitted by the sample material using a chip-based spectrometer; processing the collected spectra with a computing device to determine a numerical difference between sample spectra and reference spectra from a training set; classifying the sample material based on the numerical difference using a machine learning model stored in the computing device; generating a unique spectral fingerprint of the sample material for authentication purposes; and providing an output indicating the authenticity of the sample material.

### BENEFIT CLAIM

This application claims the benefit under 35 U.S.C. § 119 of provisional application 63/547,070, filed Nov. 2, 2023, the entire contents of which are hereby incorporated by reference herein for all purposes as if fully set forth herein.

### FIELD OF DISCLOSURE

One technical field of the present disclosure is directed to the authentication of the molecular composition of a product or a material using spectrometry. Another technical field is directed to using the transmitted, reflected, or excited reflectance (i.e., fluorescence) of light spectra from a material and using these spectra to classify that material. Another technical field is the use of chip-based spectrometers in combination with a tuned set of light-emitting diodes in an apparatus with enhanced detection of absorption and fluorescence spectra of a product or a material. Another technical field is constructing a unique graphic pattern from the data gleaned from such an apparatus that may serve as a spectral “fingerprint.”

### BACKGROUND

The approaches described in this section are approaches that could be pursued but not necessarily approaches that have been previously conceived or pursued. Therefore, unless otherwise indicated, it should not be assumed that any of the approaches described in this section qualify as prior art merely by their inclusion in this section.

A consumer purchasing a product typically trusts that the product is authentic, that the product's manufacturing process is well managed, that the product has been stored properly, and that the product has not been contaminated or modified during a distribution process. Unfortunately, while hoping to buy an authentic product, a consumer may be purchasing a product that is counterfeit, contaminated, improperly stored, or modified in some manner. Hence, a means to verify that a product is authentic, with a cost commensurate with the value of the product, is needed and useful. Therefore, the need for authentication and classification of manufactured products or materials is significant, as many forged products are on the market.

However, the currently available systems for classifying a material at the molecular level are expensive and require expert operators. Some costly devices, such as UV spectrometers, UV-fluorescence spectrometers, Visible light spectrometers, NIR spectrometers, FTIR spectrometers, or NMR scanners, may be required to classify a given material. The selection and operation of these devices for a specific compound will rely on expert knowledge of the devices and the compound's molecular structure. Such services are expensive, and how and where products may be authenticated and classified is limited.

Advancements in the creation of optical sensors, spectral sensors, and spectrometers on a chip have led to the availability of sensors useful for the authentication and classification of specific materials and products.

Additionally, a variety of light sources for these sensors is expanding due to the growth of new methods of constructing Light Emitting Diodes using new materials, dopants, and tunable fluorescent components. Fluorescent components such as Quantum Dots in Quantum Dot Light Emitting Diodes provide tunable, very narrow line spectra for use in a new generation of optical devices. Such sources are directly compatible with the digital IO of processors containing a complete system on a chip (SOC) and cost far less than legacy full spectrum or line spectrum sources.

Furthermore, the availability of low-cost support for machine learning models processed locally in the hardware of mobile devices or on the new class of SOCs will also affect the quality and availability of software to classify the light spectra obtained from a material's reflectance, transmission, or excited reflectance.

However, those systems are usually unable to use the area of the image sensors optimally. They cannot classify a specific set of materials or map each spectrum to an area of the image sensor. Therefore, there is a need to develop technologies that rely on the fundamental changes, the new class of on-chip sensors, the new classes of line spectrum and full spectrum light emitting diode (LED) light sources, and the new hardware support of machine learning models and open-source software for classifiers built for this hardware.

### BRIEF DESCRIPTION OF THE DRAWINGS

The patent or application file contains at least one drawing executed in color. The Office will provide copies of this patent or patent application publication with color drawings upon request and payment of the necessary fee.

The way the above-recited features of the present disclosure can be understood in detail, briefly summarized above, is that a more particular description of the disclosure may be had by reference to aspects, some of which are illustrated in the appended drawings. It is to be noted, however, that the appended drawings illustrate only typical aspects of this present disclosure and are therefore not to be considered limiting of its scope, for the present disclosure may admit to other equally practical aspects.

In the drawings:

FIG. 1 shows raw reference transmission spectra of distilled water illuminated by 12 LEDS.

FIG. 2 shows a calibration curve of a chip spectrometer with a wavelength in nanometers on the y-axis and the index of the sample on the x-axis.

FIG. 3 shows the raw spectra of a sample of red wine using the same 12 LEDs.

FIG. 4 shows the adjusted absorption spectra of the red wine sample in FIG. 3.

FIG. 5 shows the raw reference of the 395 nm LED.

FIG. 6 shows a red wine's fluorescent response from the reference's excitation input from FIG. 5.

FIG. 7 shows the spectra of 16 Red wines sorted based on the two wines with the most considerable classifier distance from one another.

FIG. 8 shows a listing of the 16 Red wines in FIG. 7 and their distance from the two red wines chosen as the first and last based on their maximum distance within the set of wines.

FIG. 9 shows a comparison of the 16 red wines with the full spectrum of each.

FIG. 10 is a listing in order of the 16 red wines shown in FIG. 9.

FIG. 11 shows the adjusted spectra of each of the 12 transmission LEDs with the spectra clipped to show only samples where the distilled water reference amplitude is 5% or higher of the maximum distilled water reference amplitude for that LED's spectrum.

FIG. 12 shows a list of the spectra in FIG. 11 sorted by the number of sample entries in each spectrum.

FIG. 13 shows a fingerprint constructed from the sorted spectra list in FIG. 11 and FIG. 12.

FIG. 14 shows a daughterboard that holds the LED sources for transmission spectra.

FIG. 15 shows a left-side view of the assembled apparatus without a cuvette to hold the red wine sample.

FIG. 16 shows a left-side view of the assembled apparatus with a cuvette to hold the red wine sample, excluding the temperature sensor daughterboard (for clarity).

FIG. 17 shows an LED reflection daughterboard.

FIG. 18 shows a perspective view of the assembled apparatus with a cuvette to hold the red wine sample.

FIG. 19 shows a separate perspective view of the assembled apparatus without a cuvette to hold the red wine sample.

FIG. 20 is an example flow chart of a process for authenticating and classifying products using hyper-spectral imaging.

FIG. 21 is an example flow chart of a process for authenticating and classifying products using hyper-spectral imaging.

FIG. 22 is a block diagram depicting a computer system upon which some embodiments may be implemented.

### DESCRIPTION OF EXAMPLE EMBODIMENTS

The following description outlines numerous details to understand the present approach thoroughly. It will be apparent, however, that the present approach may be practiced without these specific details. In other instances, well-known structures and devices are shown in block diagram form to avoid unnecessarily obscuring the present approach.

Embodiments are described herein according to the following outline:
1.0. GENERAL OVERVIEW
   1.1. AUTHENTICATING AND CLASSIFYING PRODUCTS
   1.2. TECHNICAL PROBLEM AND TECHNICAL SOLUTION
   1.3. TECHNICAL EFFECTS
2.0. IDENTIFYING A SET OF MATERIALS FOR CLASSIFICATION
3.0. DETERMINING A TRAINING SET OF MATERIALS
4.0. RECORDING FULL SPECTRA OF AN EXAMPLE MATERIAL
5.0. CLASSIFYING A SET OF MATERIALS BY FULL SPECTRA
6.0. DETERMINING CHARACTERISTIC LINE SPECTRA FOR THE CLASSIFICATION OF EACH MEMBER IN A SET OF MATERIALS
7.0. BUILDING A SOFTWARE CLASSIFIER BASED ON CHARACTERISTIC LINE SPECTRA
8.0. DETERMINING REQUIREMENTS FOR LIGHT SOURCES AND SENSORS
9.0. DETERMINING THE GEOMETRY OF AN APPARATUS FOR PHYSICALLY SUPPORTING, ILLUMINATING, AND SENSING A SET OF PRODUCTS OR MATERIALS
10.0. DESIGNING AND TESTING AN APPARATUS
   10.1. PHYSICAL RELATIONSHIPS BETWEEN COMPONENTS OF AN APPARATUS
   10.2. DESCRIPTION OF COMPONENTS
      10.2.1. CHIP-BASED SPECTROMETER DAUGHTERBOARD
      10.2.2. LED TRANSMISSION DAUGHTERBOARD
      10.2.3. LED REFLECTION DAUGHTERBOARD
      10.2.4. TEMPERATURE SENSOR DAUGHTERBOARD
      10.2.5. SYSTEM ON A CHIP (SOC) MICROCONTROLLER ASSEMBLY
      10.2.6. MOTHERBOARD
11.0. OPERATIONAL DESCRIPTION OF AN APPARATUS
12.0. CALIBRATION OF EXPOSURE TIMING
13.0. TAKING AND ADJUSTING A SAMPLE
14.0. CLASSIFYING A SAMPLE
15.0. DESIGNING A UNIQUE FINGERPRINT FOR MATERIALS IN A SET
16.0. FIRST EXAMPLE FLOW CHART
17.0. SECOND EXAMPLE FLOW CHART
18.0. IMPLEMENTATION MECHANISMS

### 1.0. General Overview

The technologies described here rely on several novel approaches, including a new class of on-chip sensors, new classes of line spectrum and full spectrum LED light sources, and the new hardware support of machine learning models and open-source software for classifiers built for this hardware.

The methods and systems presented herein are used to enhance and improve the process of reading the light spectra that are either transmitted by or reflected from an object or material, classifying these spectra to identify or authenticate the object or material and provide a graphic representation of the spectra that is unique to that object or material.

The methods disclosed herein rely on fundamental changes in illumination, sensing, and classification to assemble a system for the authentication and classification of molecular compositions of products or materials.

The presented methods have broad applicability in many industries for the authentication and classification of products. For example, these methods may be broadly used in fraud detection, such as the detection of counterfeited products. The presented approaches may prevent consumers from purchasing products that have been forged, contaminated, improperly stored, or otherwise modified improperly. Usually, the product consumers trust the sellers and believe that the product manufacturing processes have been well managed and that the products have been appropriately stored and protected from contamination or improper distribution. However, that trust may be insufficient to guarantee that the products are authentic.

The presented methods allow for determining the authenticity of the products and determining that the product is commensurate with the product's actual value. The approaches are relatively easy for consumers to implement and use.

Among other things, the approaches rely on several fundamental changes made to fabricating the illuminants for spectral analysis, the sensors for spectral analysis, and changes in how the elements operate and perform the classification. These newly available illuminants, sensors, and classification means are used to assemble a novel system for the authentication and classification of the molecular composition of a product or a material.

In some implementations, the presented approach allows for the simultaneous sensing of selected narrow wavelengths of the light spectra rather than a full continuous spectrum.

The disclosed systems and methods are used to optimize the use of illuminants, spectral sensors, and hardware-based classifiers for authentication and fingerprinting of a selected set of objects or materials.

### 1.1. Authenticating and Classifying Products

The present approaches introduce novel systems and methods for authenticating and classifying products using hyper-spectral imaging. The key novelties include, among other things, the Integration of Chip-Based Spectrometers. It utilizes chip-based spectrometers combined with a tuned set of light-emitting diodes (LEDs) to enhance the detection of absorption and fluorescence spectra.

The approaches are also cost-effective and user-friendly. They provide a more affordable and user-friendly alternative to traditional spectrometry systems, which are typically expensive and require expert operators.

Furthermore, they provide a Machine Learning Integration. They incorporate machine learning models processed locally on mobile devices or system-on-chip (SOC) hardware to classify light spectra efficiently.

Moreover, they provide a Unique Spectral Fingerprint. They construct a unique graphic pattern or “fingerprint” from the spectral data, allowing for easy identification and authentication of materials.

In addition, they utilize Versatile Light Sources. They employ a variety of LED light sources, including those with narrow line spectra, to provide comprehensive spectral analysis.

Furthermore, the approaches utilize the Optimized Sensor Usage. More specifically, they address limitations in current systems by optimizing the use of image sensors to classify specific materials and map spectra to sensor areas.

These improvements collectively enhance the ability to authenticate and classify materials at a molecular level in a cost-effective and accessible manner.

### 1.2. Technical Problem and Technical Solution

The need to authenticate and classify manufactured products or materials remains significant. Consumers often face the risk of purchasing counterfeit products, items that have been contaminated, improperly stored, or modified. Consumers rely on the assumption that product manufacturing is well managed, storage conditions are optimal, and distribution processes are secure. A reliable means to verify product authenticity, with a cost proportional to the product's value, is both necessary and beneficial.

Existing systems for material classification at the molecular level are costly and require expert operation. Devices such as UV, UV-fluorescence, and Visible light spectrometers, NIR spectrometers, FTIR spectrometers, and NMR scanners are often necessary for material classification. The selection and operation of these devices demand expert knowledge of both the devices and the molecular structure of the compounds. This expertise incurs significant expense, limiting the accessibility and practicality of product authentication and classification.

The disclosed methods and apparatuses address these challenges by providing a cost-effective and user-friendly solution for authenticating and classifying materials. The approach utilizes advancements in optical sensors, spectral sensors, and spectrometers on a chip combined with a tuned set of light-emitting diodes. This system enhances the detection of absorption and fluorescence spectra, enabling the construction of a graphic pattern that serves as a spectral fingerprint. The method leverages new technologies to optimize the use of illuminants, spectral sensors, and hardware-based classifiers, facilitating the authentication and fingerprinting of selected objects or materials.

### 1.3. Technical Effects

The present methods utilize a plurality of light-emitting diodes (LEDs) to illuminate both training and sample substances, allowing for the collection of spectra using a spectrometer. This setup enables the detection of specific spectral characteristics of the substances, which can be used to determine their molecular composition. LEDs provide a cost-effective and versatile light source compared to traditional light sources for spectrometry, which are often expensive and require expert operation.

By determining a numerical difference between the spectra of training substances and a sample substance, the methods allow for the classification of the sample substance. This approach provides a practical application for authenticating materials by comparing their spectral data against known references, thus addressing the need for a reliable and accessible means of verifying product authenticity.

Integrating a processor to compute the numerical difference and classify the sample substance streamlines the process, making it feasible for non-expert users to authenticate materials. This reduces the dependency on specialized equipment and expertise while broadening the methods' applicability across various industries where material verification is critical.

### 2.0. Identifying a Set of Materials for Classification

In some implementations, an approach presented herein facilitates the enhancement and improvement of reading the light that an object transmits or reflects. Analyzing the spectral light lines is essential for various reasons, including characterizing objects' composition, colors, surfaces, and an object or material's fluorescence. For example, the analysis may include characterizing a piece of fine leather or red wine.

The analysis of the spectral lines may be used to, for example, identify and/or verify the authenticity of materials. This may include verifying the authenticity of, for example, the leather used in a Gucci® handbag or the authenticity of wine in a vintage wine bottle. Furthermore, the analysis of the spectral lines may be used to distinguish red wine from one geographical region and red wine from another area.

The spectral lines may be used to, for example, verify the distinct characteristics of wine. The characteristics may be determined based on the different and separate spectra that can be separated across the light spectra. For instance, red wine may be characterized by the information included in the UV light spectrum, the visible spectrum, and the Near IR spectrum.

### 3.0. Determining a Training Set of Materials

In this disclosure, material may refer to a substance, compound, or product within a set that the system must classify or authenticate. The analysis of the spectral lines transmitted through or reflected from, for example, wine may include determining the wine characteristics that indicate the presence or absence of and the amount of, for instance, water, tannic acid, fructose, Gallic acid or ethanol, and other wine components that absorb specific spectra of light. The training materials for red and white wines may include a set of reagent chemicals in concentrations in water similar to that found in red wines. The training set may also include specific varieties of red wines for classification. These red wines may include Cabernet Sauvignon, Pinot Noir, Barolo, Rose, and others. The training set may include various wines from specific regions, such as a Cabernet Sauvignon from Napa Valley in California and a Cabernet Sauvignon from Australia. The training set may include specific authentication wines, such as a Screaming Eagle Cabernet Sauvignon from Napa Valley in California.

### 4.0. Recording Full Spectra of an Example Material

In some implementations, the Near UV, Visible, and Near IR spectra may be collected for each item in the training set. For instance, these spectra may be collected using a Thor Labs CCS200 Extended range spectrometer, an array of light emitting diodes (LEDs) selected to provide, in aggregate, full illumination over the extended range of the CCS200. The LED array may illuminate a sample of wine held by a cuvette (i.e., a type of sample holder for liquid samples), the light may be transmitted through the sample, and the CCS200 may read the extended spectrum. These spectra may be organized and assembled into a training set for classification.

Additionally, an array of LEDs may be selected to provide reflected illumination of the sample. These may be chosen to offer line sources known to stimulate fluorescence in active components within the set of materials.

### 5.0. Classifying a Set of Materials by Full Spectra

The training set collected above may be filtered for training in several steps.

In some implementations, the spectra of the aggregate light sources are collected with a neutral target (such as distilled water in the case of red wine or a titanium white matte surface in the case of leather) so that they may be used to characterize the aggregate light sources. For instance, for each data collection session for a specific item in the training set, a series of spectra of distilled water may be obtained. These spectra may be used to normalize the recorded spectra of the samples for that session.

In some implementations, a statistical median distilled water reference spectrum is chosen as a reference by finding the median of the spectra. One method for finding the median is to sort many spectra of the distilled water sample by their closeness to the two reference spectra that are furthest from each other in the distance, as calculated by the Least Squares method. The reference median corresponds to the sample halfway through the list of samples sorted by distance. Next, the median spectrum of the spectra taken of an object or material in the training set may be chosen similarly. Finally, normalizing the item's spectrum in the training set may be performed by scaling that spectrum's amplitude by the reciprocal amplitude of the reference distilled water spectra. As a practical matter, the reciprocal may be limited if the amplitude reference distilled water sample for that spectral line is below a certain threshold. In one instance, this threshold may be chosen as 5% of the maximum value of all samples in the reference.

Next, the normalized spectra for each item in the training set may be filtered for noise. In some implementations, this may be performed by applying a Pseudo Gaussian IIR filter to the amplitude of the normalized spectrum. The device's resolution may determine the Gaussian filter's window or size. In this case, the CCS200 has a resolution of about two (2) nm and records a spectral line about every 0.5 nm so that the Gaussian window may be set to three (3).

Finally, each spectrum may be compressed by the presence of high-frequency features. That is, the areas with more minor changes in amplitude may be sampled sparsely, while the areas with significant changes in amplitude may be preserved.

In some implementations, the filtered spectrum data for each item in the training set may be used to build a tensor flow model to match an unknown spectrum to a specific varietal of wine.

In some situations, the filtered spectrum data for each item in the training set may be used to build a Support Vector Machine model to determine how close an unknown spectrum is to a specific product release.

Alternatively, the filtered spectrum data for each item in the training set may be used to build a Scale Invariant Feature Transform model to determine how close an unknown spectrum is to a specific product release.

Furthermore, the filtered spectrum data for each item in the training set may be used to build a Partial Least Squares model to determine how close an unknown spectrum is to a specific product release.

### 6.0. Determining Characteristic Line Spectra for the Classification of Each Member in a Set of Materials

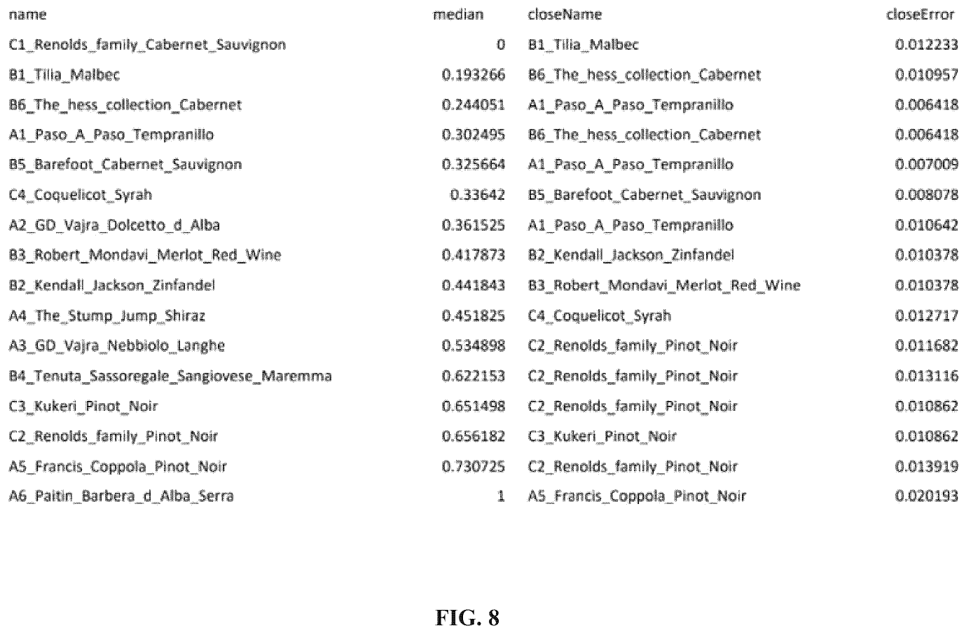

The classifier constructed as described in the previous section may be used to determine a numerical difference between spectra collected from one material in the set from another. These differences may be used to automatically filter for pairs of spectra that are close to one another (see FIG. 8, the closeName column—described later). Complete sessions of these close pairs may be compared by differencing. The resulting differenced spectra may be used for Principal Component Analysis. The portions of the spectrum most helpful in separating close pairs may be accumulated. These will be the characteristic spectra.

### 7.0. Building a Software Classifier Based on Characteristic Line Spectra

A new classifier may be built and trained with the training set created as described above. The training set may be filtered to use only the characteristic portions of the spectrum. It may be validated by testing its classification using data withheld from the training set. It may also be tested by applying noise to the original training set (e.g., noise determined based on the range and classified difference for a given sample session from which the median was taken).

### 8.0. Determining Requirements for Light Sources and Sensors

The present approach may utilize various sensors. However, the most suitable sensors are hyper-spectral imaging sensors and on-chip spectrometers.

The characteristic portions of the spectrum determined using sensors, as described above, may be used to determine the requirements for the light sources and the sensors. When applied to individual light sources, it may be used to determine which spectra of which light sources may provide a means of separating two or more materials from one another and which may provide a means for determining if a light source is to be used in constructing the apparatus for classifying the set of objects or materials. An S-chip-based grating spectrometer, a chip-based hyper-spectral imaging system, or a chip-based FTIR spectrometer may be chosen for constructing the apparatus for classifying the set of objects or material based on the coverage of valuable spectrum regions for the classifier.

### 9.0. Determining the Geometry of an Apparatus for Physically Supporting, Illuminating, and Sensing a Set of Products or Materials

The initial geometry of the apparatus may be based on the optical qualities of the set of products or materials. If the materials are transparent (e.g., red wine), then the apparatus will need to support a transmitted light geometry for sensing the absorption spectrum of that material. If the material is fully opaque, the apparatus will need to support a reflected light geometry to sense the absorption spectrum of that material. Both sets of material will require separate reflection geometry to sense the fluorescent response of the material. Additionally, the apparatus will need to hold a sample of the material at a constant distance from the sensor and light sources to obtain reproducible results. Additionally, the apparatus will need to restrict ambient light from the environment so that it does not read the sensor or interfere with the illumination of the material sample.

If the material changes the absorption of fluorescent characteristics based on temperature, the apparatus will need a means to control the temperature of the sample. If the LED light illumination or the sensor spectra are affected by temperature, the apparatus will need a means to control the ambient temperature of the device.

### 10.0. Designing and Testing an Apparatus

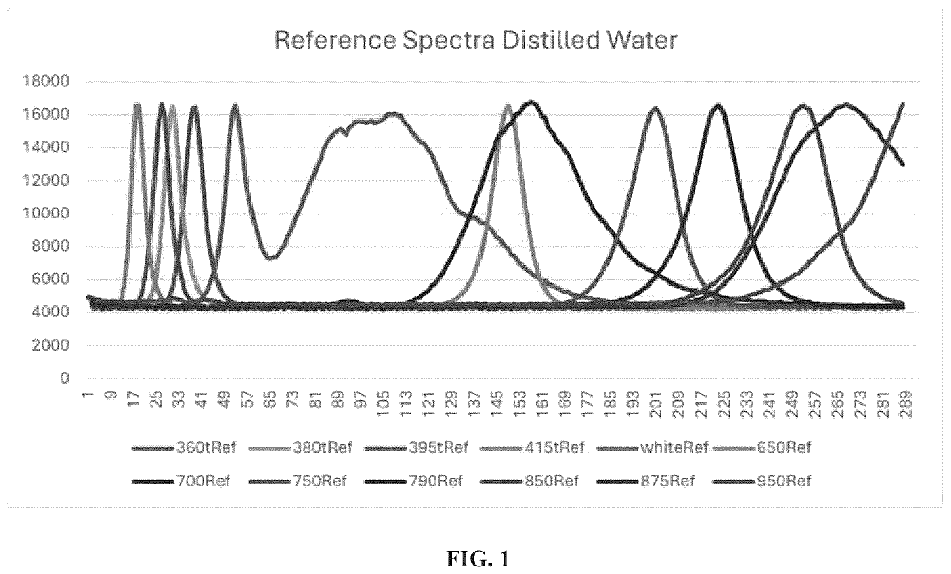

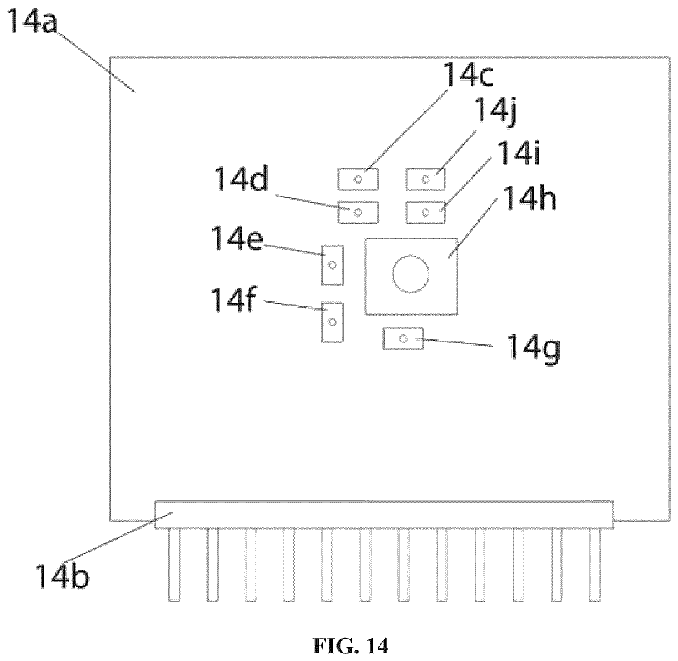

The design for an apparatus for authenticating and providing a fingerprint for red wine based on the above-described requirements is shown in FIG. 14 through FIG. 19 (described later). The resulting output of an apparatus built from this design is shown in FIG. 1 through FIG. 13 (described later).

### 10.1. Physical Relationships Between Components of an Apparatus

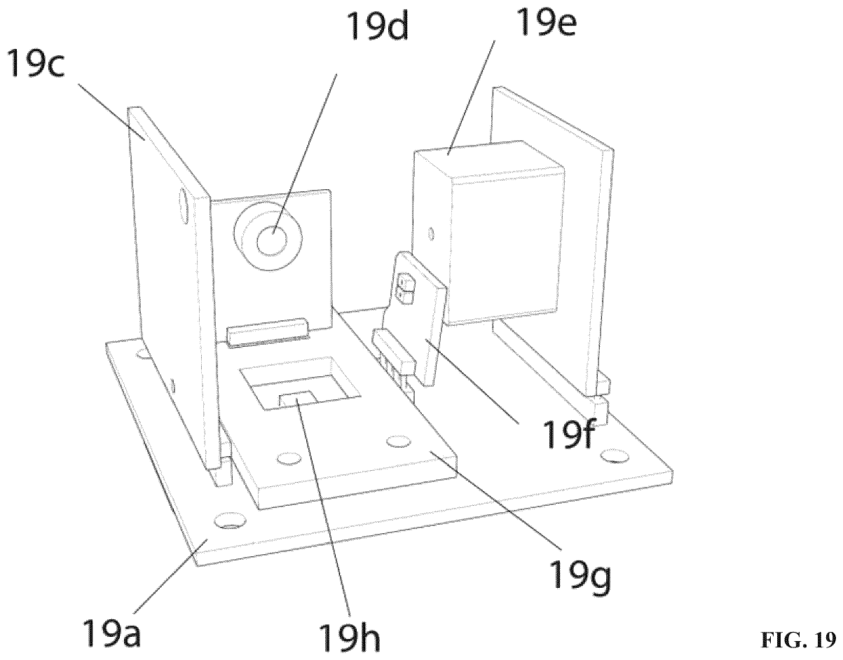

FIG. 19 shows a separate perspective view of the assembled apparatus without a cuvette to hold the red wine sample. In FIG. 19, motherboard 19a provides a regulated power backplane with the daughterboards and the alignment fixture for a cuvette. It has the SOC microprocessor and provides a digital backplane for controlling the LED daughterboard and the NuvVizNir Spectrometer daughterboard.

FIG. 19 also shows a temperature sensor daughterboard 19d, a NuvVizNir Spectrometer daughterboard 19e, which holds a chip-based spectrophotometer ranging from 300 to 900 nm. FIG. 19 also shows an LED reflection daughterboard 19f, an alignment fixture 19g with a cutout that fits closely to hold the cuvette, and a resistive heater 19h that is controlled by the SOC microprocessor to provide a constant temperature for the cuvette above the ambient room temperature.

As shown in FIG. 19, motherboard 19a supports and holds LED transmission daughterboard 19c, non-contact temperature sensor daughterboard 19d, a chip-based spectrometer 19e, LED reflection daughterboard 19f, alignment fixture 19g, and resistive heating element 19h.

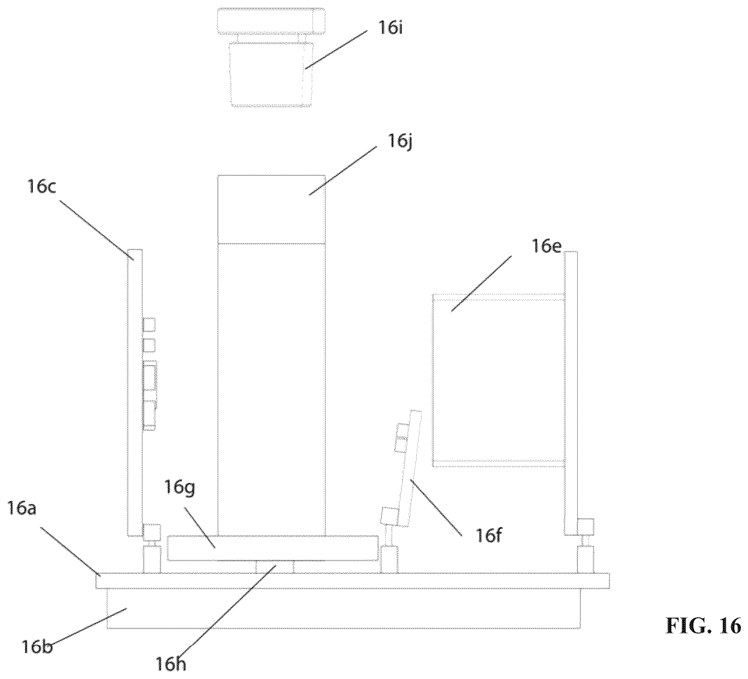

FIG. 16 shows a left-side view of the assembled apparatus with a cuvette to hold the red wine sample, excluding the temperature sensor daughterboard (for clarity). FIG. 16 shows a motherboard 16a, which provides a regulated power backplane, holds the daughter boards and the alignment fixture for the cuvette, holds the SOC microprocessor assembly, and provides a digital backplane for controlling the LED daughterboard and the NuvVizNir Spectrometer daughterboard. 16b is the SOC microprocessor, which runs firmware to communicate with external devices (such as a mobile phone), reads the temperature sensor daughterboard, and controls the timing for the LED transmission daughterboard and the NuvVizNir Spectrometer daughterboard. 16c is the LED transmission daughterboard, which is also described in FIG. 14. A NuvVizNir Spectrometer daughterboard 16e holds a chip-based spectrophotometer ranging from 300 to 900 nm. 16f is the LED reflection daughterboard. 16g is an alignment fixture with a cutout that fits closely to hold the cuvette. 16h is a resistive heater controlled by the SOC microprocessor to provide a constant temperature for the cuvette above the ambient room temperature. 16j is a quartz cuvette used to hold the wine sample. 16i is a stopper for the cuvette.

As shown in FIG. 16, motherboard 16a supports 16b and the SOC microcontroller assembly. As also shown in FIG. 16, the apparatus is designed to transmit a series of light impulses from LED transmission daughterboard 16c, through cuvette 16j holding a red wine sample, to chip-based spectrometer 16e.

The apparatus also may transmit a series of light pulses from LED reflection daughterboard 16f to stimulate a fluorescent emission from cuvette 16j containing a red wine sample, which falls on chip-based spectrometer 16e.

### Example Assembled Apparatus

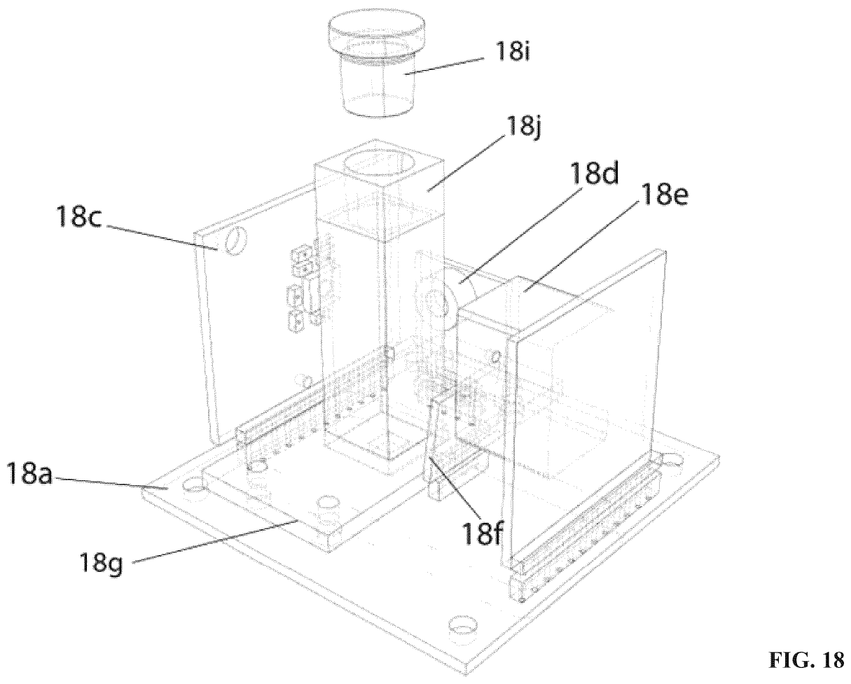

FIG. 18 shows a perspective view of the assembled apparatus with a cuvette to hold the red wine sample. In FIG. 18, 18a is the motherboard, which provides a regulated power backplane, holds the daughter boards and the alignment fixture for the cuvette, holds the SOC microprocessor, and provides a digital backplane for controlling the LED daughterboard and the NuvVizNir Spectrometer daughterboard. 18d is the temperature sensor daughter board. 18e is the NuvVizNir Spectrometer daughterboard; it holds a chip-based spectrophotometer ranging from 300 to 900 nm. 18f is the LED reflection daughterboard. 18g is an alignment fixture with a cut-out that fits closely to hold the cuvette. 18j is a quartz cuvette for holding the wine sample. 18i is the stopper for the cuvette.

In FIG. 18, an alignment fixture 18g closely holds cuvette 18j holding a red wine sample so that it may be placed repeatable in the same position relative to LED transmission daughterboard 18c, LED reflection daughterboard 18f, chip-based spectrometer 18e, and non-contact temperature sensor daughterboard 18d.

Referring again to FIG. 19, cuvette 19g of red wine has been removed for clarity, and an alignment fixture may closely hold the cuvette containing a red wine sample in direct contact with a resistive heating element 19h and in repeatable placement with a non-contact temperature sensor daughterboard 19d.

### 10.2. Description of Components

### 10.2.1. Chip-Based Spectrometer Daughterboard

The spectrometer daughterboard holds a chip-based spectrometer located relative to the cuvette containing the red wine sample, the LED Transmission Daughterboard, and the LED Reflection Daughterboard. It provides protection and voltage adjustments for the serial data lines on the spectrometer chip, voltage regulation for the spectrometer chip, and analog signal filtering for the analog signal from the spectrometer chip if needed. The chip-based spectrometer provides analog outputs serially over the sensed spectrum based on a timing line. As noted above, the chip-based spectrometer may be chosen based on its spectral range and resolution compared to the many available spectrometers. Hamamatsu Corporation makes several such devices. The one chosen for this design is the C12880MA.

### 10.2.2. LED Transmission Daughterboard

FIG. 14 shows a daughterboard that holds the LED sources for transmission spectra. In the depicted example, the spectra are transmitted through a cuvette holder and received by the spectrometer chip. 14a is the printed circuit board that holds the LEDs and connects them to the SOC microcontroller through 14b. 14b is a pin header that provides the connection to the microcontroller and the physical mounting and position of the board. 14c is a narrow spectrum Near UV 365 nm LED. 14d is a narrow spectrum Near UV 395 nm LED. 14e is a moderately narrow spectrum Near IR 850 nm LED. 14f is a broad spectrum visible warm white LED at 4000 Kelvin. 14g is a moderately broad spectrum Near IR 875 nm LED. 14h is a multi-LED chip with moderately narrow spectra at 600 nm, 750 nm, 790 nm, 850 nm, and 950 nm. 14i is a moderately broad spectrum Visible Red 700 nm LED. 14j is a narrow spectrum Near UV 385 nm LED. The output of these LEDs is shown in FIG. 1.

### Reference Transmission Spectra

FIG. 1 shows raw reference transmission spectra of distilled water illuminated by 12 LEDs. FIG. 1 shows direct readings of a sensor's raw reference transmission spectra. Difference reference spectra are color-coded and include, for example, a 360t reference, a 380t reference, a 395t reference, and so forth.

### Calibration Curve

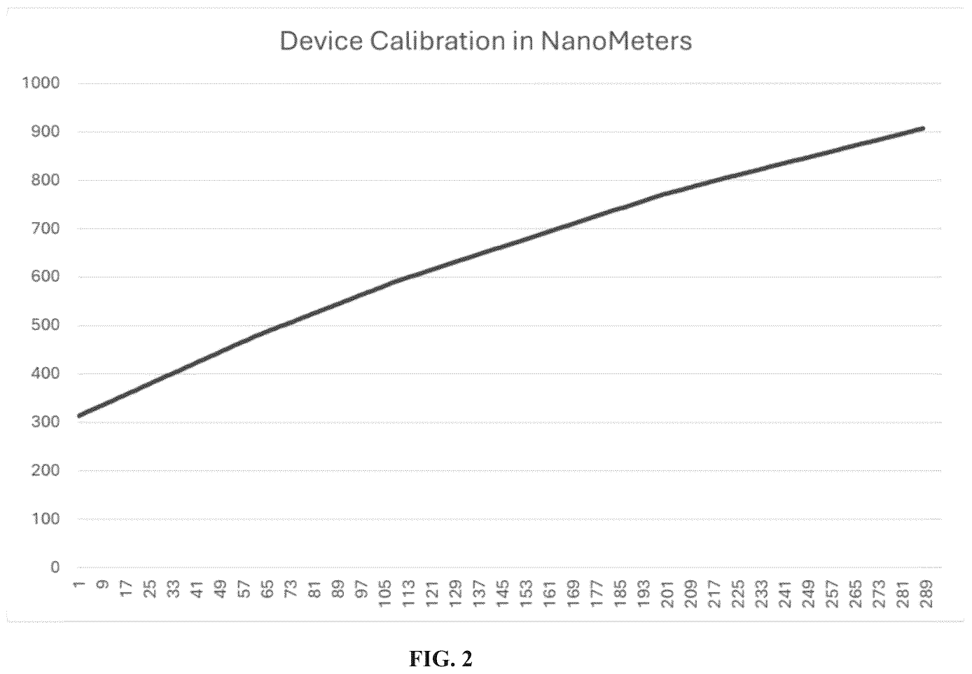

FIG. 2 shows a calibration curve of a chip spectrometer with a wavelength in nanometers on the y-axis and the index of the sample on the x-axis. It should be noted that the relationship between the calibration curve in the y-direction is proportional to that in the x-direction.

### Raw Spectra

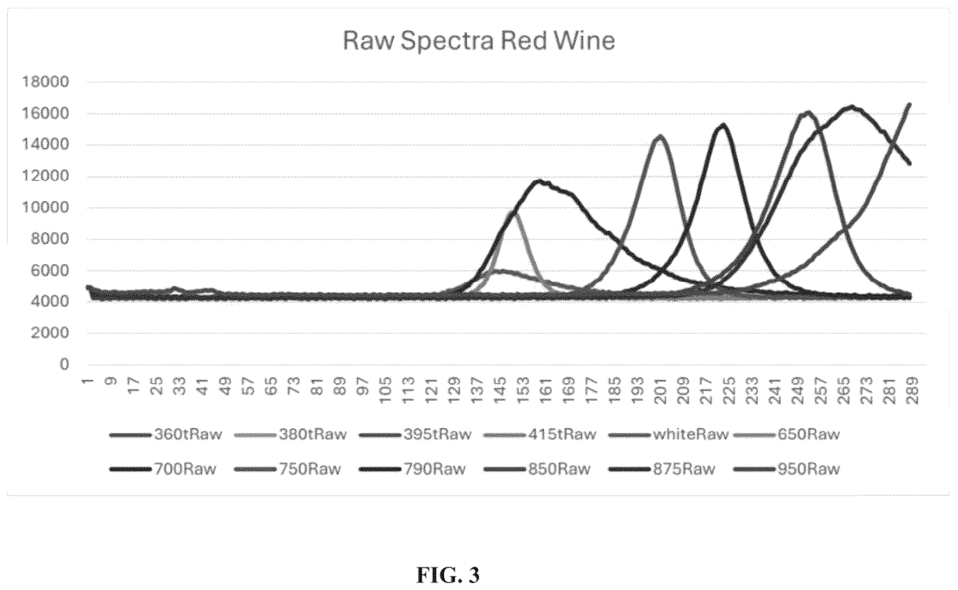

FIG. 3 shows the raw spectra of a sample of red wine using the same 12 LEDs. More specifically, FIG. 3 shows direct readings from a sensor. The raw spectra are color-coded and include, for example, a 360t raw, a 380t raw, a 395t raw, and so forth.

### Adjusted Absorption Spectra

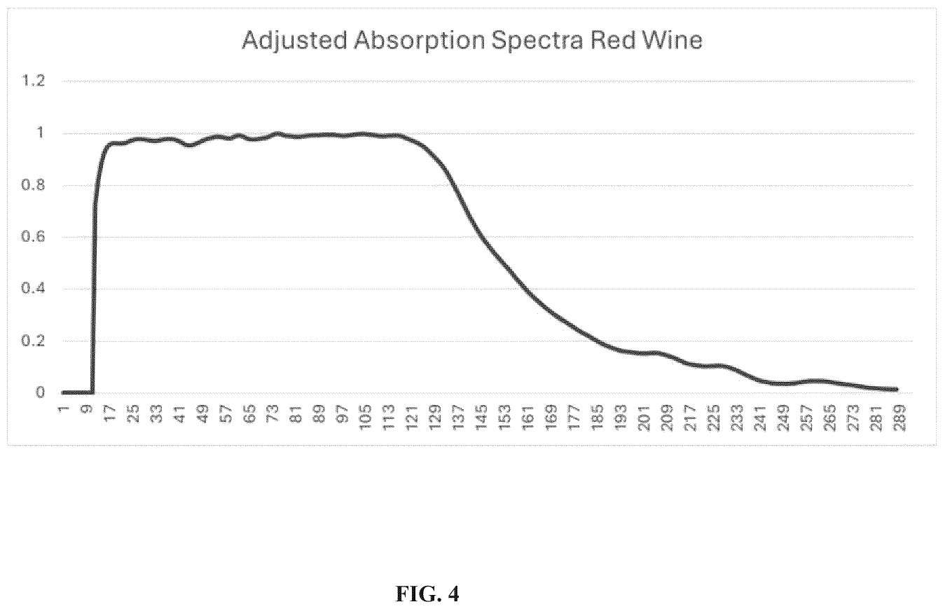

FIG. 4 shows the adjusted absorption spectra of the red wine sample in FIG. 3. More specifically, FIG. 4 shows the absorption spectra adjusted using the reference transmission spectra of distilled water to calculate the absorption of light from 0 . . . 1.

### Example of Relatively Narrow Spectrum

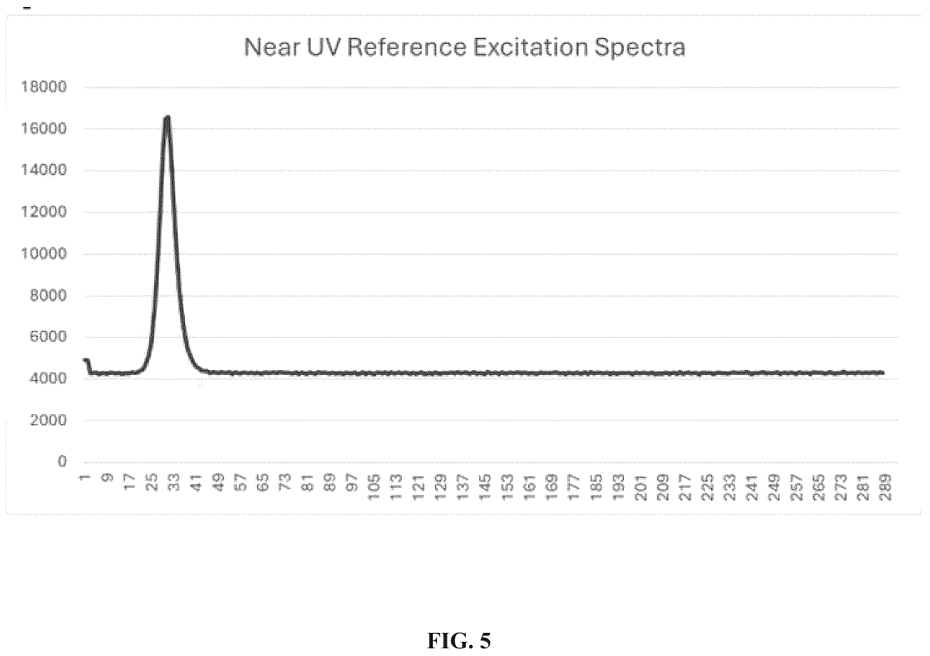

FIG. 5 shows the raw reference of the 395 nm LED. It should be noted that this is an example of a relatively narrow spectrum, and the reading is a direct reading from the sensor.

### Red Wine's Fluorescent Response

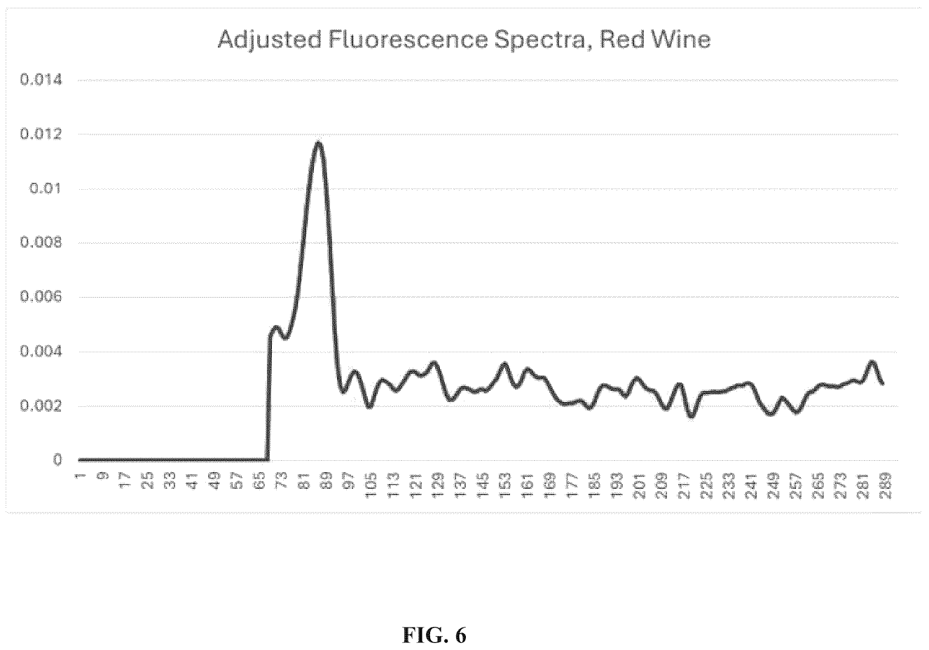

FIG. 6 shows a red wine's fluorescent response from the reference's excitation input from FIG. 5. In this example, the response has been normalized to the input amplitude of that reference. It should be noted that the fluorescent output is frequency-shifted.

### Spectra of Example Wines

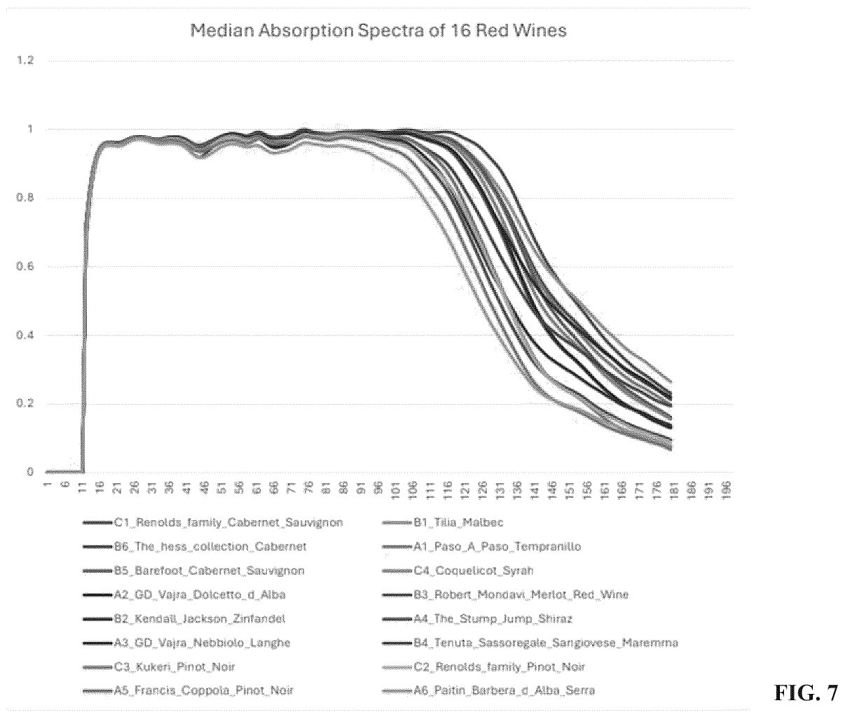

FIG. 7 shows the spectra of 16 Red wines sorted based on the two wines with the most considerable classifier distance from one another. In this example, the spectra were clamped to 730 nm to reduce temperature change errors. The different wines are color-coded, as shown in FIG. 7.

### Listing of Example Wines

FIG. 8 shows a listing of the 16 Red wines in FIG. 7 and their distance from the two red wines chosen as the first and last based on their maximum distance within the set of wines. The example list shows the closest match to each wine, the error of the closest match, and the error from the first wine in the list. The distance is 0 . . . 1 as that maximum distance normalizes it. The median distance may be found by choosing the red wine in the center of the list.

### Example Comparison of Red Wines

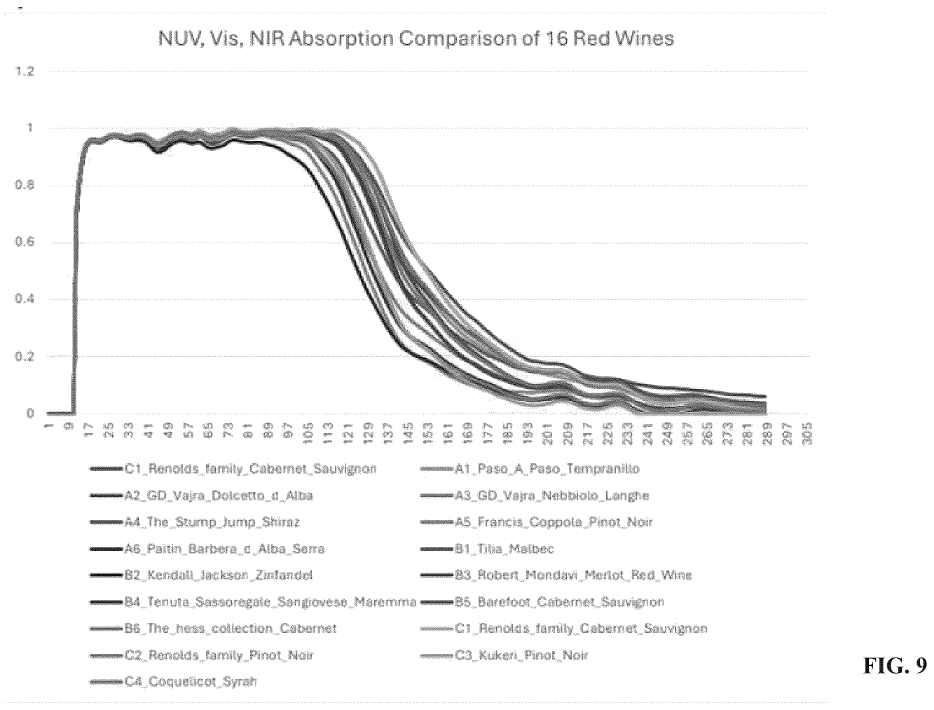

FIG. 9 shows a comparison of the 16 red wines with the full spectrum of each. The different wines are color-coded, as shown in FIG. 9.

### Example of an Ordered List of Red Wines

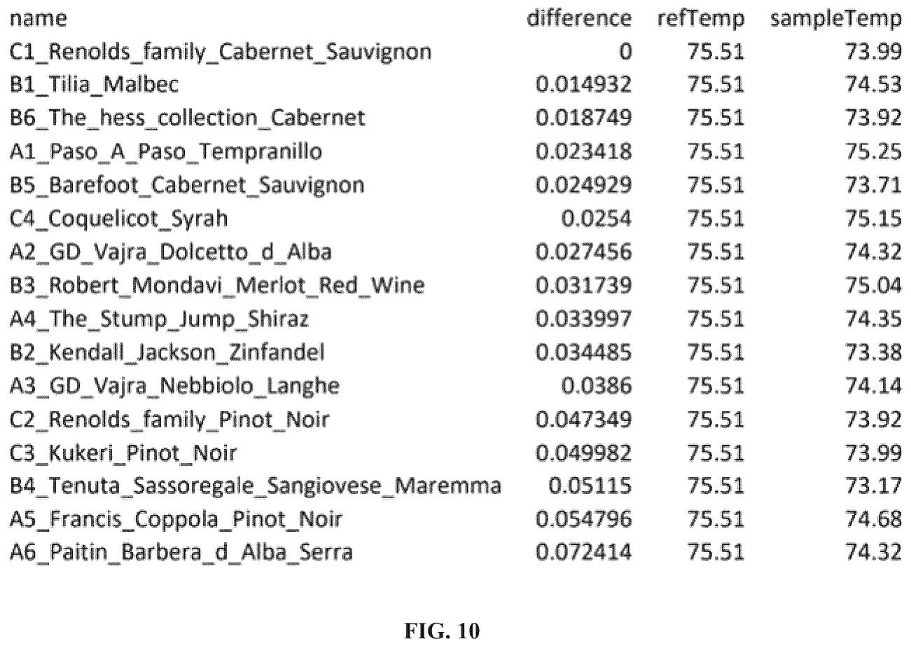

FIG. 10 is a listing in order of the 16 red wines shown in FIG. 9. The distance value from the first wine in the list is determined by a classifier, the temperature of the distilled water reference used to adjust the sample, and the temperature of the sample.

### Example of Adjusted Spectra

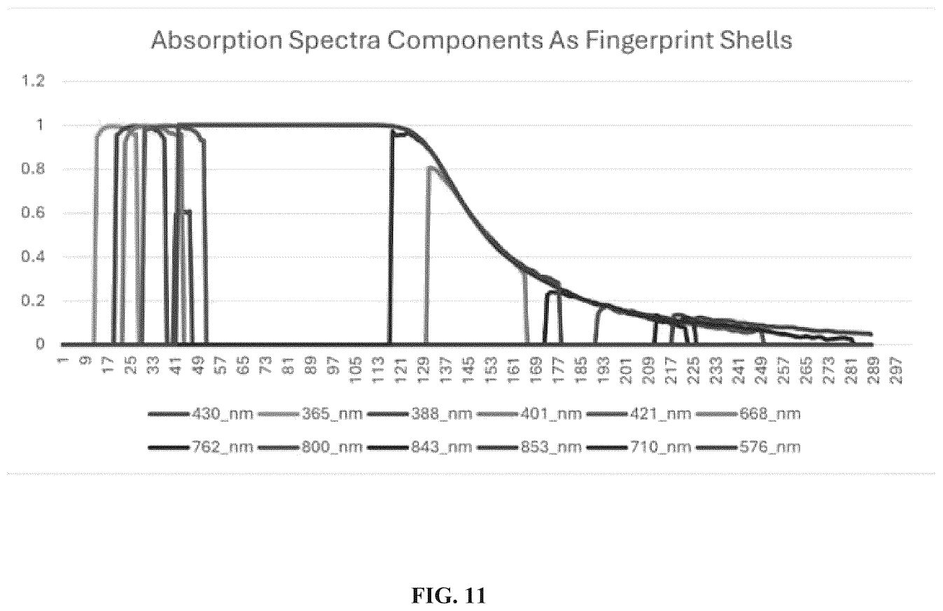

FIG. 11 shows the adjusted spectra of each of the 12 transmission LEDs with the spectra clipped to show only samples where the distilled water reference amplitude is 5% or higher of the maximum distilled water reference amplitude for that LED's spectrum. The different spectra are color-coded and shown in FIG. 11. Additional information about the adjusted spectra is provided later.

### Example List of Spectra

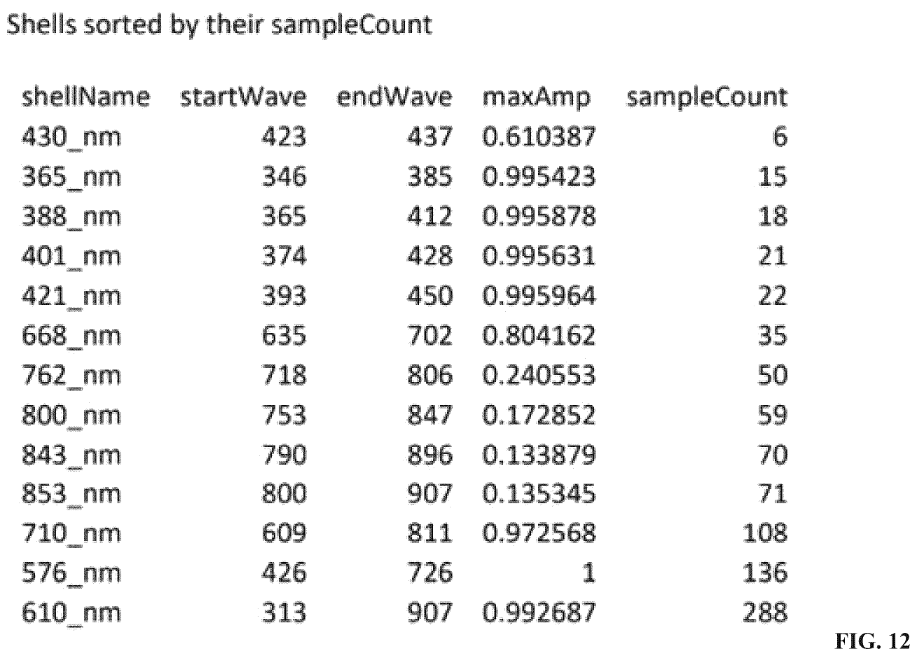

FIG. 12 shows a list of the spectra in FIG. 11 sorted by the number of sample entries in each spectrum. Additional information about the spectra is provided later.

### Example Fingerprint

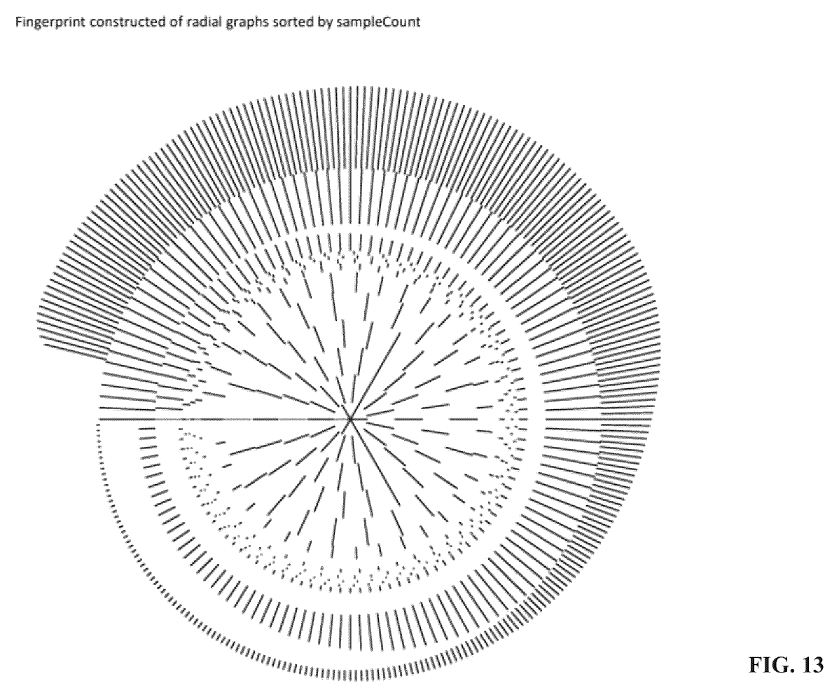

FIG. 13 shows a fingerprint constructed from the sorted spectra list in FIGS. 11 and 12. In the depicted example, each spectrum is rendered as a radial line graph from the center of the fingerprint to the outside. The spectra in FIG. 12 with the smallest number of samples are rendered first. FIG. 12 serves as a key for the fingerprint. Additional information about the fingerprints is provided later.

Referring again to FIG. 14, 14a is the daughterboard, a printed circuit board that holds the LEDs and connects them to the SOC microcontroller through 14b. The daughterboard also has attachment holes for further physical support by an enclosure.

14b is a pin header that provides the connection to the microcontroller and the physical mounting and position of the board. It provides a data line to control each LED on the daughterboard and power connection as a return for the data line.

14c is a narrow spectrum Near UV 365 nm LED. One example of such a chip is the Bivar SM1206UV-365-IL.

14d is a narrow spectrum Near UV 395 nm LED. One example of such a chip is the Bivar SM1206UV-395-IL.

14e is a moderately narrow spectrum Near IR 850 nm LED. One example of such a chip is the Kingbright APTD3216SF4C-P22.

14f is a broad spectrum visible warm white LED at 4000 Kelvin. One example of such a chip is the Bivar SM1206UWC-IL.

14g is a multi-LED chip with moderately narrow spectra at 600 nm, 750 nm, 790 nm, 850 nm, and 950 nm. One example of such a chip is the Marktech MTMD6788594SMT6.

14i is a moderately broad spectrum Visible Red 700 nm LED. One example of such a chip is the Bivar SM0805RC.

14j is a narrow spectrum Near UV 385 nm LED. One example of such a chip is the Kingbright ATS2012UV385.

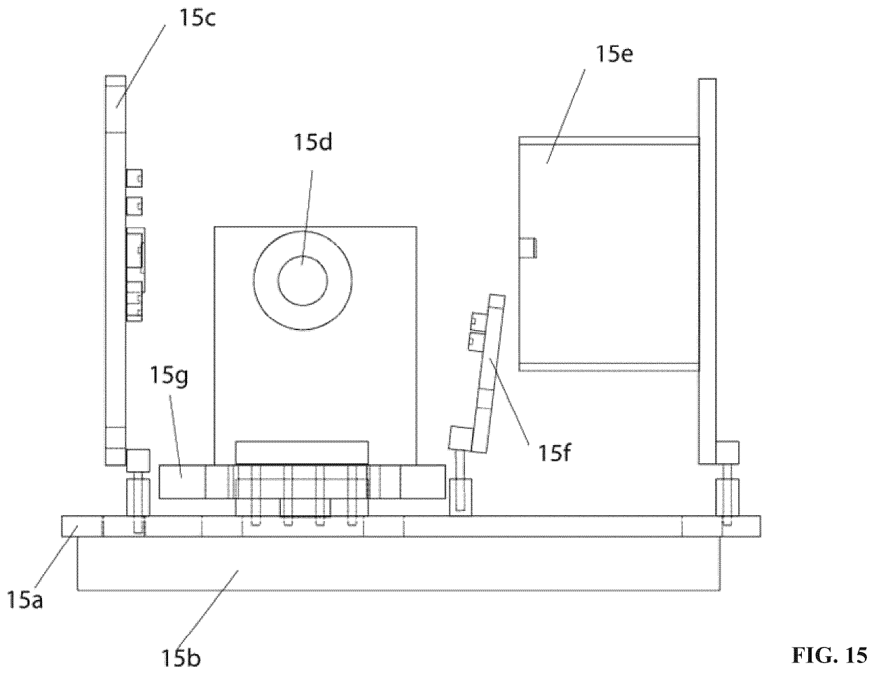

FIG. 15 shows a left-side view of the assembled apparatus without a cuvette to hold the red wine sample. In FIG. 15, 15a is the motherboard, which provides a regulated power backplane, has the daughter boards and the alignment fixture for the cuvette, holds the SOC microprocessor assembly, and provides a digital backplane for controlling the LED daughterboard and the NuvVizNir Spectrometer daughterboard.

15b is the SOC microprocessor assembly, which runs firmware to communicate with external devices (such as a mobile phone), reads the temperature sensor daughterboard, and controls the timing for the LED transmission daughterboard and the NuvVizNir Spectrometer daughterboard.

15c is the LED transmission daughterboard described in FIG. 14.

15d is the temperature sensor daughterboard.

15e is the NuvVizNir Spectrometer daughterboard; it holds a chip-based spectrophotometer ranging from 300 to 900 nm.

15f is the LED reflection daughterboard.

15g is a holder which provides physical alignment for the cuvette.

FIG. 16 shows a left-side view of the assembled apparatus with a cuvette to hold the red wine sample, excluding the temperature sensor daughterboard (for clarity).

16a is the motherboard, which provides a regulated power backplane, has the daughter boards and the alignment fixture for the cuvette, holds the SOC microprocessor assembly, and provides a digital backplane for controlling the LED daughterboard and the NuvVizNir Spectrometer daughterboard.

16b is the SOC microprocessor assembly, which runs firmware to communicate with external devices (such as a mobile phone), reads the temperature sensor daughterboard, and controls the timing for the LED transmission daughterboard and the NuvVizNir Spectrometer daughterboard.

16c is the LED transmission daughterboard described in FIG. 14.

16e is the NuvVizNir Spectrometer daughterboard; it holds a chip-based spectrophotometer ranging from 300 to 900 nm.

16f is the LED reflection daughterboard.

16g is a holder which provides physical alignment for the cuvette.

16h is a heating element controlling the temperature of the cuvette.

16i is the stopper of the cuvette

16j is the cuvette which holds the material to be authenticated.

### 10.2.3. LED Reflection Daughterboard

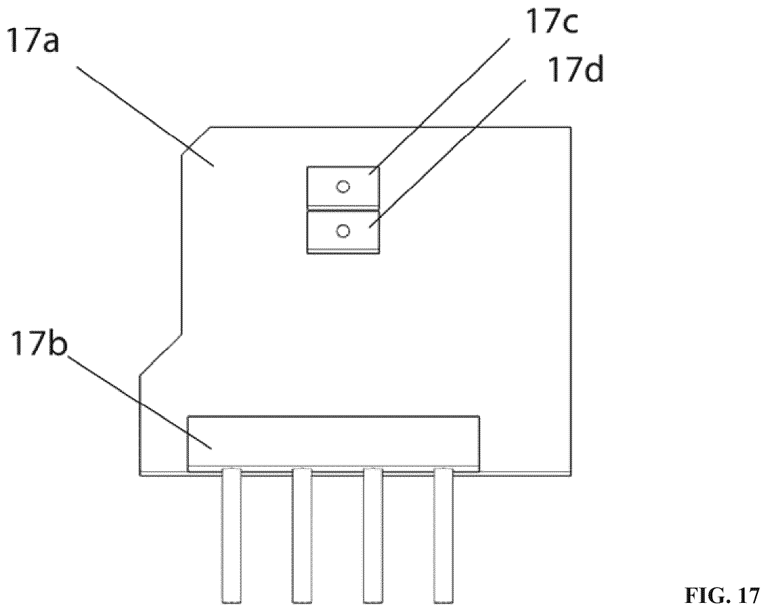

FIG. 17 shows an LED reflection daughterboard. It illuminates the cuvette with Near UV light to excite compounds within the red wine to fluoresce.

17a is the printed circuit daughterboard that holds the LEDs and connects them to the SOC microcontroller through 17b.

17b is a pin header that provides the connection to the microcontroller and the physical mounting and position of the board; it is adjusted to give a 30-degree angle of illumination relative to the light input for the spectrometer. It provides a data line to control each LED on the daughterboard and power connection as a return for the data line.

17c is a narrow spectrum Near UV 415 nm LED. One example of such a chip is the Bivar SM1206UV-400-IL.

17d is a narrow spectrum Near UV 395 nm LED. One example of such a chip is the Bivar SM1206UV-395-IL.

### 10.2.4. Temperature Sensor Daughterboard

A non-contact Temperature Sensor Daughterboard holds a non-contact temperature sensor, provides a physical mounting and alignment of the sensor relative to the cuvette, and provides a connection to a Serial System Management Bus and power. One example of a non-contact temperature sensor is the Melexis MLX90614. The sensor offers an ambient device temperature and a non-contact cuvette temperature over the SMB bus. The SOC microcontroller uses it to manage temperature.

### 10.2.5. System on a Chip (SOC) Microcontroller Assembly

The SOC Microcontroller Assembly includes a System on a Chip Microcontroller with analog to Digital lines, Digital IO lines, and USB serial communications. The added components of the assembly provide wireless communication via Bluetooth or Wi-Fi, a power source, and power regulation. Many such devices are available depending on the processing requirements and if the SOC assembly handles a machine learning model and hardware computation or is resident on a mobile device through wireless communication.

### 10.2.6. Motherboard

A motherboard provides a physical alignment and mounting of the daughterboards, alignment fixture, and resistive heating element. It also provides data and power connections to the SOC Microcontroller Assembly components.

### 11.0. Operational Description of an Apparatus

In some implementations, the apparatus performs a series of specific and well-timed actions to calibrate and take a single sample of Red Wine. The calibration actions are taken in two separate significant steps. The first is calibrating the exposure timing for each LED light source; the second is acquiring a reference spectrum for each LED light source. Both require placing a cuvette holding distilled water in the alignment fixture.

### 12.0. Calibration of Exposure Timing

A SOC microcontroller may control the exposure of the spectrometer to each LED light by timing when the trigger data line for the spectrometer's data acquisition is held high and continues high (within fixed constraints). In contrast, a specific LED data line is pulled low (to light the LED), and a timing pulse is started. The exposure continues until the LED line returns to high and the trigger data line is pulled low, at which time the exposure is done. The timing pulse continues for a fixed number of pulses; then, the serial analog output is read and stored by the SOC as each timing pulse continues until the entire spectrum is read.

The orchestration of these timed events must be precise. They can be performed by timing microcontroller instructions if the microcontroller has few interrupts and does not cache instructions. Supporting these timed events in software can reduce the physical chips needed to control the C12880MA. Careful choice of the SOC microcontroller, such as using the Microchip SAM 32-bit Cortex family, can reduce complexity and cost.

The timing instructions may be encoded in a sub-routine and called as needed. It is necessary to calibrate the timing for each LED separately because the output of the LED will vary based on its physical characteristics and placement. Additionally, the spectrometer's detector array will respond differently to different wavelengths. One approach to calibrating the exposure time is determining the best maximum value of the returned signal. In the case of the C12880MA, this is about 80% of the clipped or saturated exposure value. Once this number is determined, it is possible to write a software function that solves the exposure time iteratively for a given LED to return the best maximum value at its spectral peak.

Referring again to FIG. 1, it shows the spectrum of each LEDs once they have been calibrated. It should be noted that all their maximum peaks are closely aligned. The exposure value for each LED may be stored in the SOC's non-volatile memory.

An application on a mobile device may provide a user interface to allow a user to calibrate the apparatus. The mobile device may establish a communication stream with the apparatus using a wireless protocol. The mobile device may request that the SOC calibrate and store the calibrated exposure values.

### 13.0. Taking and Adjusting a Sample

The actions required to take a reference sample for calibration and a sample of red wine may be the same until the reference sample is used to normalize or adjust the red wine sample.

A user interface to allow a user to take a reference or a red wine sample may be provided by an application on a mobile device. The application on the mobile device may provide an interface showing the user how to place a sample in the apparatus. The mobile device application may provide a user interface allowing the user to start sample collection. The mobile device may establish a communication stream with the apparatus using a wireless protocol. The mobile device may request that the SOC perform the sample.

The apparatus may call the subroutine to perform an exposure with each LED in turn, using the calibrated exposure time for that LED. Each LED Spectrum may be stored in the local memory of the SOC. In the case of a reference sample (e.g., distilled water), each spectrum may be stored in non-volatile memory. Once all the spectra are taken in the case of a red wine sample, each one may be adjusted or normalized using the stored reference sample. Referring again to FIG. 11, it shows a graph of the adjusted spectra. Additionally, the reference samples may be accumulated into a combined reference. The red wine sample may be accumulated into a combined sample, and the combined reference may be used to adjust or normalize the combined sample. FIG. 4 shows an adjusted combined spectrum for a red wine sample.

### 14.0. Classifying a Sample

After adjusting the spectra, they may be used as input to classify or authenticate the wine. The classifier and its stored model data may reside in non-volatile memory in the SOC. In this case, the authentication or match may be found using the resident classifier and associated model, and the result is returned to the mobile device in response to its request.

### 15.0. Designing a Unique Fingerprint for Materials in a Set

A unique fingerprint representing a sample of red wine (or other sample of a product or material that the apparatus is designed to authenticate) may be constructed in the following manner:

1. The spectra for each LED exposure and the combined spectra may be ordered based on the sample count (the number of samples in the spectrum that are not close to zero). See FIG. 12.

2. The spectra are rendered as nested radial graphs in shells ordered from the center of the radial graphs to the outside. See FIG. 13.

3. A list of the order of the spectra and their range may be provided as a key. See FIG. 12.

### 16.0. First Example Flow Chart

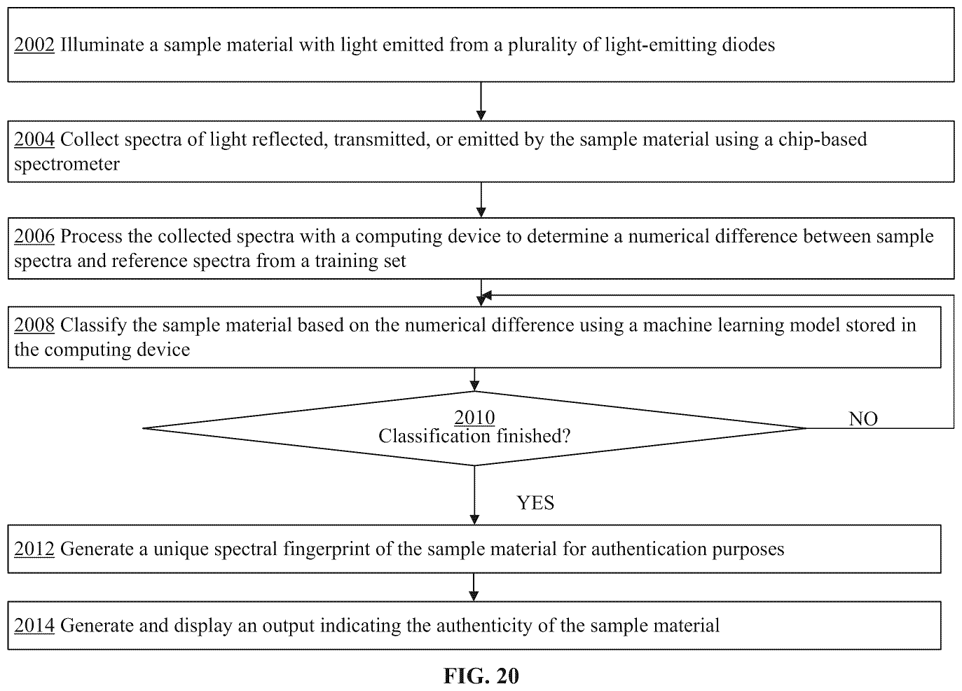

FIG. 20 is an example flow chart of a process for authenticating and classifying products using hyper-spectral imaging. The steps herein may be performed by the apparatus described above.

In step 2002, an apparatus illuminates a sample material with light emitted from a plurality of light-emitting diodes (LEDs). This may be performed to determine a set of characteristics of light spectra reflected or transmitted by the sample material when a plurality of light wavelengths illuminates the material. The geometry for collecting that information is depicted in, for example, FIG. 15.

In step 2004, the apparatus collects spectra of light reflected, transmitted, or emitted by the sample material using a chip-based spectrometer. The spectra may be used to construct one or more classifiers configured to classify each material of the set of materials based on the set of characteristics of the light spectra.

In step 2006, the apparatus processes the collected spectra with a computing device to determine a numerical difference between sample spectra and reference spectra from a training set.

In step 2008, the apparatus classifies the sample material based on the numerical difference using a machine-learning model stored in the computing device

In step 2010, the apparatus checks if the classification has been completed successfully. If it has, then the apparatus proceeds to perform step 2112. Otherwise, the apparatus proceeds to step 2008.

In step 2012, the apparatus generates a unique spectral fingerprint of the sample material for authentication purposes.

In step 2014, the apparatus generates and displays an output indicating the authenticity of the sample material.

### 17.0. Second Example Flow Chart

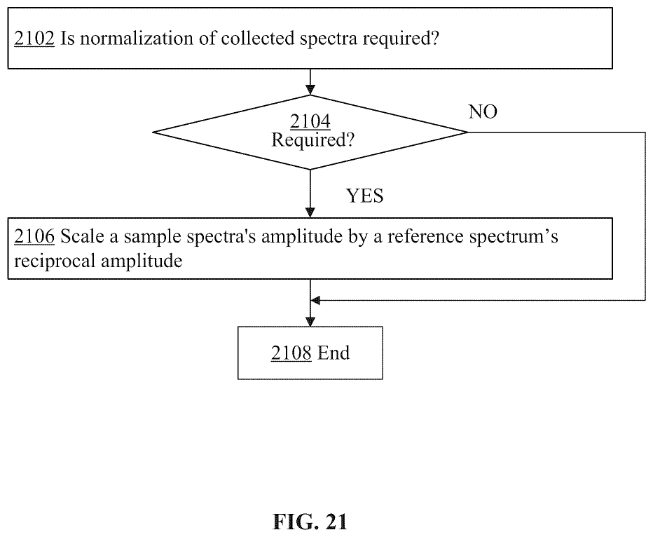

FIG. 21 is an example flow chart of a process for authenticating and classifying products using hyper-spectral imaging. The steps herein may be performed by the apparatus described above.

In step 2102, an apparatus determines whether the collected spectra need to be normalized.

If, in step 2104, the apparatus determines that normalization is needed, then the apparatus performs step 2106. Otherwise, the apparatus performs step 2108

In step 2106, the apparatus maps, based on, at least in part, the classifiers, each of the light spectra onto an area of an image sensor.

In step 2108, the apparatus normalizes the spectra. This may involve scaling a sample spectra's amplitude by a reference spectrum's reciprocal amplitude.

### 18.0. Implementations Mechanisms

Although the flow diagrams of the present application depict a particular set of steps in a specific order, other implementations may use fewer or more steps in the same or different order than those shown in the figures.

According to one embodiment, the techniques described herein are implemented by one or more special-purpose computing devices. The special-purpose computing devices may be hard-wired to perform the methods or may include digital electronic devices such as one or more application-specific integrated circuits (ASICs) or field programmable gate arrays (FPGAs) that are persistently programmed to perform the techniques or may include one or more general purpose hardware processors programmed to perform the methods pursuant to program instructions in firmware, memory, other storage, or a combination. Such special-purpose computing devices may combine custom hard-wired logic, ASICs, or FPGAs with custom programming to accomplish the techniques. The special-purpose computing devices may be desktop computer systems, portable computer systems, handheld devices, networking devices, or any other device that incorporates hard-wired and/or program logic to implement the techniques.

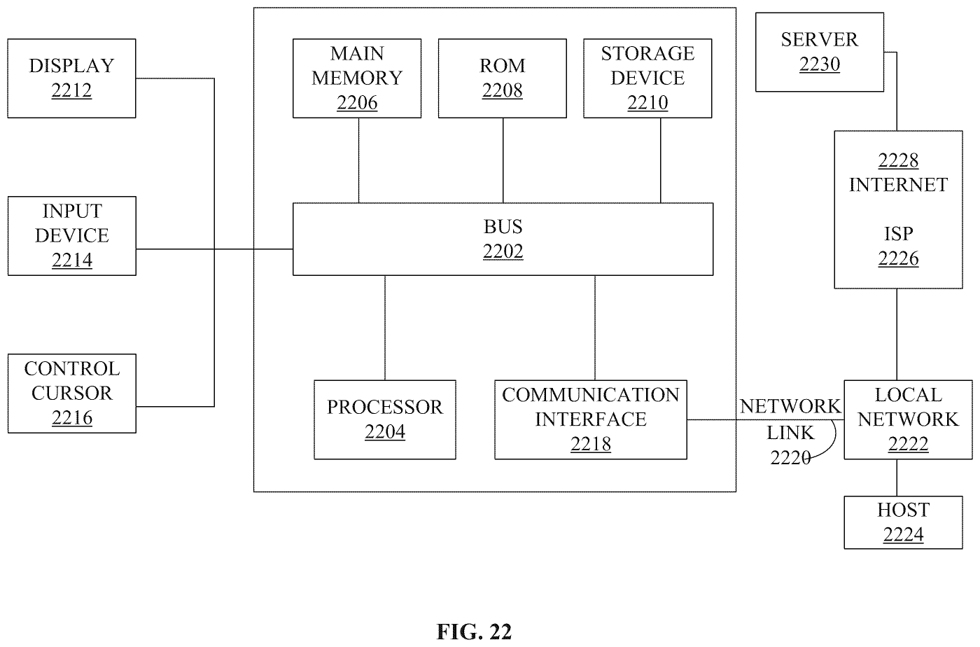

FIG. 22 is a block diagram depicting a computer system 2200 upon which some embodiments may be implemented. Computer system 2200 includes a bus 2202 or other communication mechanism for communicating information and a processor 2204 coupled with bus 2202 for processing information. Computer system 2200 also includes a main memory 2206, such as random-access memory (RAM) or another dynamic storage device coupled to bus 2202 for storing information and instructions to be executed by processor 2204. Main memory 2206 may also be used to store temporary variables or other intermediate information during the execution of instructions to be executed by processor 2204. Computer system 2200 includes a read-only memory (ROM) 2208 or other static storage device coupled to bus 2202 for storing static information and instructions for processor 2204. A storage device 2210, such as a magnetic or optical disk, is provided and coupled to bus 2202 for storing information and instructions.

Computer system 2200 may be coupled via bus 2202 to a display 2212, such as a cathode ray tube (CRT), for displaying information to a computer user. Although bus 2202 is illustrated as a single bus, bus 2202 may comprise one or more buses. For example, bus 2202 may include without limitation a control bus by which processor 2204 controls other devices within computer system 2200, an address bus by which processor 2204 specifies memory locations of instructions for execution, or any other type of bus for transferring data or signals between components of computer system 2200.

An input device 2214, including alphanumeric and other keys, is coupled to bus 2202 for communicating information and command selections to processor 2204. Another type of user input device is cursor control 2216, such as a mouse, a trackball, or cursor direction keys for communicating direction information and command selections to processor 2204 and controlling cursor movement on display 2212. This input device typically has two degrees of freedom in two axes, a first axis (e.g., x) and a second axis (e.g., y), that allows the device to specify positions in a plane.

Computer system 2200 may implement the techniques described herein using customized hard-wired logic, one or more ASICs or FPGAs, firmware and/or program logic, or computer software which, in combination with the computer system, causes or programs computer system 2200 to be a special-purpose machine. According to one embodiment, those techniques are performed by computer system 2200 in response to processor 2204 executing one or more sequences of one or more instructions contained in main memory 2206. Such instructions may be read into main memory 2206 from another computer-readable medium, such as storage device 2210. Execution of the sequences of instructions contained in main memory 2206 causes processor 2204 to perform the process steps described herein. In alternative embodiments, hard-wired circuitry may be used in place of or in combination with software instructions to implement the embodiments. Thus, embodiments are not limited to any specific combination of hardware circuitry and software.

The term “computer-readable medium” refers to any medium that provides data that causes a computer to operate in a specific manner. In an embodiment implemented using computer system 2200, various computer-readable media are involved, such as providing instructions to processor 2204 for execution. Such a medium may take many forms, including, but not limited to, non-volatile media and volatile media. Non-volatile media includes, for example, optical or magnetic disks, such as storage device 2210. Volatile media includes dynamic memory, such as main memory 2206. Typical forms of computer-readable media include, for example, a floppy disk, a flexible disk, hard disk, magnetic tape, or any other magnetic medium, a CD-ROM, any other optical medium, a RAM, a PROM, and EPROM, a FLASH-EPROM, any other memory chip, or memory cartridge, or any other medium from which a computer can read.

Various forms of computer-readable media may be involved in carrying one or more sequences of one or more instructions to processor 2204 for execution. For example, the instructions may initially be carried on a magnetic disk of a remote computer. The remote computer can load the instructions into its dynamic memory and send them over a telephone line using a modem. A modem local to computer system 2200 can receive the data on the telephone line and use an infrared transmitter to convert the data to an infrared signal. An infrared detector can receive the data from the infrared signal, and appropriate circuitry can place the data on bus 2202. Bus 2202 carries the data to main memory 2206, from which processor 2204 retrieves and executes the instructions. The instructions received by main memory 2206 may optionally be stored on storage device 2210 either before or after execution by processor 2204.

Computer system 2200 also includes a communication interface 2218 coupled to bus 2202. Communication interface 2218 provides a two-way data communication coupling to a network link 2212 connected to a local network 2222. For example, communication interface 2218 may be an integrated service digital network (ISDN) card or a modem to connect data to a corresponding telephone line. Another example is communication interface 2218, a local area network (LAN) card that provides a data communication connection to a compatible LAN. Wireless links may also be implemented. In any such implementation, communication interface 2218 sends and receives electrical, electromagnetic, or optical signals that carry digital data streams representing various types of information.

Network link 2212 typically provides data communication to other data devices through one or more networks. For example, network link 2212 may connect through local network 2222 to a host computer 2224 or data equipment operated by an Internet Service Provider (ISP) 2226. ISP 2226 provides data communication services through the worldwide packet data communication network, now commonly called the “Internet” 2228. Local networks 2222 and Internet 2228 use electrical, electromagnetic, or optical signals that carry digital data streams.

Computer system 2200 can send messages and receive data, including program code, through the network(s), network link 2212, and communication interface 2218. In the Internet example, server 2230 might transmit a requested code for an application program through Internet 2228, ISP 2226, local network 2222, and communication interface 2218. The received code may be executed by processor 2204 as it is received and/or stored in storage device 2210 or other non-volatile storage for later execution.

In the foregoing specification, embodiments have been described with reference to numerous specific details that may vary from implementation to implementation. Thus, the sole and exclusive indicator of what is, and is intended by the applicants to be, the approach is the set of claims that issue from this application in the specific form in which such claims issue, including any subsequent correction. Hence, no limitation, element, property, feature, advantage, or attribute that is not expressly recited in a claim should limit the scope of such claim in any way. Accordingly, the specification and drawings are to be regarded in an illustrative rather than a restrictive sense.

What is claimed is:

1. A method for authenticating a material using hyper-spectral imaging, the method comprising:
illuminating a sample material with light emitted from a plurality of light-emitting diodes (LEDs);
collecting spectra of light reflected, transmitted, or emitted by the sample material using a chip-based spectrometer;
processing the collected spectra with a computing device to determine a numerical difference between sample spectra and reference spectra from a training set;
classifying the sample material based on the numerical difference using a machine-learning model stored in the computing device;
generating a unique spectral fingerprint of the sample material for authentication purposes; and
generating and displaying an output indicating the authenticity of the sample material.

2. The method of claim 1, further comprises normalizing the collected spectra using a reference spectrum obtained from a neutral substance.

3. The method of claim 2, wherein the normalizing involves scaling a sample spectra's amplitude by a reference spectrum's reciprocal amplitude.

4. The method of claim 1, wherein the light emitted from the plurality of LEDs includes near-infrared, visible, or near-ultraviolet wavelengths.

5. The method of claim 1, further comprises filtering the collected spectra to reduce noise using a Gaussian filter.

6. The method of claim 1, wherein the machine learning model is a Support Vector Machine or a TensorFlow model.

7. The method of claim 1, further comprises constructing a radial graph as the unique spectral fingerprint of the sample material.

8. The method of claim 1, wherein the output indicating authenticity is provided via a user interface on a mobile device.

9. An apparatus for authenticating and classifying a material, comprising:
a sample holder configured to contain a sample;
a first plurality of light-emitting diodes (LEDs) and a spectrometer forming ends of a first optical path, wherein the spectrometer receives light emitted from the first plurality of LEDs and transmitted through the sample;
a second plurality of LEDs and the spectrometer forming ends of a second optical path, wherein light emitted from the second plurality of LEDs is reflected from the sample and directed to the spectrometer;
a temperature control system comprising a temperature sensor and a heater configured to maintain a consistent temperature of the sample; and
a computing device comprising a processor and a memory, the computing device in electronic communication with the first and second pluralities of LEDs and the spectrometer, configured to control the LEDs, receive data from the spectrometer, and process the data using machine learning models to classify and authenticate the material.

10. The apparatus of claim 9, wherein the sample holder is made of quartz for enhanced optical clarity.

11. The apparatus of claim 9, wherein the first plurality of LEDs comprises Quantum Dot LEDs for narrow line spectra.

12. The apparatus of claim 11, wherein the second plurality of LEDs also comprises Quantum Dot LEDs for narrow line spectra.

13. The apparatus of claim 9, further comprising a temperature control system with a resistive heater and a non-contact temperature sensor.

14. The apparatus of claim 9, wherein the spectrometer has a range of 300 to 900 nm.

15. The apparatus of claim 9, wherein the computing device implements a TensorFlow model for real-time classification.

16. The apparatus of claim 15, further comprising Gaussian filtering for noise reduction in spectra analysis.

17. The apparatus of claim 9, wherein the computing device uses Bluetooth or Wi-Fi for wireless communication.

18. A computer-readable storage media storing one or more computer instructions which, when executed by one or more computing processors, cause the one or more computing processors to perform:
illuminating a sample material with light emitted from a plurality of light-emitting diodes (LEDs);
collecting spectra of light reflected, transmitted, or emitted by the sample material using a chip-based spectrometer;
processing the collected spectra with a computing device to determine a numerical difference between sample spectra and reference spectra from a training set;
classifying the sample material based on the numerical difference using a machine learning model stored in the computing device;
generating a unique spectral fingerprint of the sample material for authentication purposes; and
providing an output indicating the authenticity of the sample material.

19. The computer-readable storage media of claim 18, storing additional instructions for: normalizing the collected spectra using a reference spectrum obtained from a neutral substance.

20. The computer-readable storage media of claim 19, wherein the normalizing involves scaling a sample spectra's amplitude by a reference spectrum's reciprocal amplitude.
